(19) United States
(12) Patent Application Publication
(10) Pub. No.: US 2012/0046552 A1
(43) Pub. Date: Feb. 23, 2012

(54) ULTRASONIC DIAGNOSTIC APPARATUS, ULTRASONIC PROBE, AND ULTRASONIC DIAGNOSTIC METHOD

(75) Inventors: Mitsuhiro Oshiki, Tokyo (JP); Kenji Maio, Tokyo (JP)

(73) Assignee: HITACHI MEDICAL CORPORATION, Tokyo (JP)

(21) Appl. No.: 13/254,743

(22) PCT Filed: Mar. 1, 2010
(86) PCT No.: PCT/JP2010/053215

§ 371 Date: Nov. 8, 2011

(30) Foreign Application Priority Data
Mar. 4, 2009 (JP) 2009-050859

(51) Int. Cl. A61B 8/14

(52) U.S. Cl. 600/459

(57) ABSTRACT

In order to provide an ultrasonic diagnostic apparatus with a transceiver circuit configuration which has a small number of components and is suitable for miniaturization, an amplifier circuit used for both transmission and reception which can be built in an ultrasonic probe and has a function of adding currents of received signals from plural elements is provided in the ultrasonic diagnostic apparatus. In addition, an analog matrix switch (402) for arbitrarily adding received signals from plural transducers is provided. That is, a transmitted and received signal amplifier circuit is shared by using a transceiver circuit section, which is formed by an FET element serving as a source follower circuit, as a gate-grounded amplifier during a signal reception period. The received signals from the transducers which have been amplified by FET elements can be added for each group of arbitrary elements by the matrix current switch (402) formed by plural FET elements corresponding to the respective transducers.

### TECHNICAL FIELD

The present invention relates to an ultrasonic diagnostic apparatus and a receiver circuit which amplifies a signal from a subject received by an ultrasonic transducer and in particular, to an ultrasonic diagnostic technique including a circuit which has both a transmission and reception separating function and a transmitted signal amplifying function.

### RELATED ART

An ultrasonic diagnostic apparatus is mainly formed of a piezoelectric material, and an electric signal is applied to transducers arrayed in the shape of a straight line or a specific curve. In addition, an element group corresponding to transmission/reception wave size is selected, and signal transmission and reception are performed while scanning them sequentially. Various kinds of information are extracted from the reflected signals to acquire information regarding the inside of a subject in a non-invasive manner.

Conventionally, a transmitter circuit which applies a voltage to transducers which have 100 or more elements is provided in the apparatus body. Generally, the number of transmitter circuits is equivalent to a phasing size of tens of channels. Ideally, independent transmitted and received signals are needed for all transducers. However, if a transmitter circuit and a receiver circuit are used for each element, the required number of components, the length of a signal cable, and the number of signal cables increase, which is not practical. For this reason, in a conventional apparatus, a configuration is adopted in which the number of transceiver circuits used corresponds to no more than the transmission/reception wave size and these transceiver circuits and transducers are connected to each other through switches (Patent Document 1).

The switch used in such a configuration turns on and off a high-voltage pulse signal at the time of signal transmission. At the same time, it is necessary to turn on and off an analog signal with a low level at the time of signal reception. Therefore, a high voltage characteristic, a function of switching a pulse signal with a large peak current value at high speed, and a low noise characteristic of low ON resistance are simultaneously required for the switching element. In addition, as a transceiver device, it is common to use a transmission and reception separating circuit for protecting a received signal amplifier from the high voltage at the time of signal transmission. As a result, there have been problems regarding the power consumption of each amplifier circuit and the transmission and reception separating circuit, an increase in the number of components, and heat emission and the mounting area.

On the other hand, Patent Document 2 realizes the miniaturization by forming a transmitted signal amplifier circuit with a switch configuration or the like. In addition, Patent Document 3 discloses adding received signals from plural transducers by a matrix switch type current adding circuit after voltage-current conversion of the received signals from the transducers. In addition, Patent Document 4 proposes an ultrasonic apparatus which has a circuit configuration suitable for variable control of a required aperture, control of scanning, and the like for plural transducers arrayed, which does not require a special device for protection against a high voltage, and which has a circuit configuration suitable for integration.

### Citation List

### Patent Documents

[Patent Document 1] JP-UM-A-56-73809

[Patent Document 2] U.S. Pat. No. 5,997,479

[Patent Document 3] JP-A-2007-185529

[Patent Document 4] JP-B-8-3528

### SUMMARY OF INVENTION

### Problem to be Solved by the Invention

In a transceiver circuit of the ultrasonic diagnostic apparatus in each of the conventional examples described above, the circuit configuration including an amplifier circuit or the like is separately set. Therefore, since there are a large number of components, miniaturization is not realized.

In addition, when using an ultrasonic probe including plural transducers, a current addition matrix circuit for adding received signals from plural arbitrary transducers is necessary. In the related art, however, a received signal amplifier circuit and an adding circuit are separately provided. Accordingly, a configuration, such as a voltage-current conversion circuit, is required before the current adding circuit.

It is an object of the invention to provide a transceiver circuit which can be built in an ultrasonic probe head that has a small number of components and is suitable for miniaturization, an ultrasonic diagnostic apparatus using it, and its ultrasonic probe.

### Solution to Problem

In order to achieve the above-described object, an ultrasonic diagnostic apparatus of the invention is configured to include: an ultrasonic probe having plural transducers; an apparatus body for reception processing of received signals from the plural transducers; a transmitted and received signal amplifier circuit which amplifies signals transmitted to the plural transducers and amplifies received signals from the plural transducers; and a current adding circuit which adds currents of the received signals amplified by the transmitted and received signal amplifier circuit.

In addition, a current adding switch which connects the transmitted and received signal amplifier circuit and the current adding circuit to each other is provided.

In addition, the transmitted and received signal amplifier circuit, the current adding circuit, and the current adding switch are built in the ultrasonic probe.

In addition, the current adding switch is formed by one FET element corresponding to each of the transducers.

In addition, an ultrasonic diagnostic apparatus is configured to include: an ultrasonic probe having plural transducers; an apparatus body for reception processing of received signals from the plural transducers; and a transmitted and received signal amplifier circuit which amplifies signals transmitted to the plural transducers and amplifies received signals from the plural transducers. The transmitted and received signal amplifier circuit includes an FET element operating as a source follower circuit at the time of signal transmission and operating as a gate-grounded circuit at the time of signal reception.

That is, in the ultrasonic diagnostic apparatus according to a first aspect of the invention, amplifier circuits used for both transmission and reception are provided in the ultrasonic probe in the relationship of 1:1 with the number of transducers so that driving of all elements is realized without using matrix switches with different amplitude characteristics for transmission and reception of signals even if the number of elements used for sound field formation like a matrix array is larger than the number of phasing channels of the ultrasonic diagnostic apparatus body.

More specifically, the transmitted and received signal amplifier circuit is shared by using a transmitter circuit, which is formed by a source follower circuit, as a gate-grounded amplifier during a signal receiving period. A transmitter circuit is formed only by a MOS field effect transistor (FET), for example, and has a simple configuration which can be formed by an existing semiconductor manufacturing process. As a result, a size capable of being built in a general ultrasonic probe head is realized.

In addition, according to a second aspect of the invention, a received signal amplifier circuit for each transducer can amplify the current of a received signal from the transducer.

In addition, according to a third aspect, a current switch is connected to the received signal amplifier circuit so that the currents can be added by grouping the received signals. The current switch group has a matrix form, and switches the number of which corresponds to the number of elements can be connected to each receiving circuit output terminal. This switch is formed by a MOSFET. One MOSFET may be provided at each connection point of the matrix, and may have a small size for switching only a received wave current.

### Advantageous Effects of Invention

As described above, according to the invention, a configuration of an amplifier circuit used for both transmission and reception which can be built in an ultrasonic probe and which has a function of adding currents of received signals from plural elements becomes possible in the ultrasonic diagnostic apparatus. According to this configuration, since the same transistor element can be used for both transmission and reception, a transmission and reception separating circuit which is grounded for protection of a receiver circuit in the related art can be eliminated. As a result, it is possible to realize a reduction in the area and an improvement in the S/N ratio.

In addition, since the gate potential of a transistor element is fixed at the time of signal reception, the input impedance is low. Accordingly, noise of the gate-grounded amplifier can be suppressed to be low.

In addition, the current-amplified received signals from each element can be added for each group of arbitrary elements by a matrix current switch.

The number of switching elements, such as MOSFETs, at each connection point of the matrix may be 1, and the switching element may have a small size for switching only a received signal current.

### BRIEF DESCRIPTION OF DRAWINGS

FIG. 1 is a block diagram of an ultrasonic diagnostic apparatus according to a first embodiment.

FIG. 2 is a view showing an example of an amplifier circuit used for both transmission and reception according to the first embodiment.

FIG. 3 is a view showing a specific example of an amplifier circuit used for both transmission and reception according to the first embodiment.

FIG. 4 is a view showing a received current adding switch according to a second embodiment.

FIG. 5 is a view showing a received current adding switch according to the second embodiment.

FIG. 6 is a block diagram of an ultrasonic diagnostic apparatus according to a third embodiment.

### DESCRIPTION OF EMBODIMENTS

Hereinafter, various kinds of embodiments of the invention will be described with reference to the drawings. In addition, it is needless to say that this does not limit the invention.

### First Embodiment

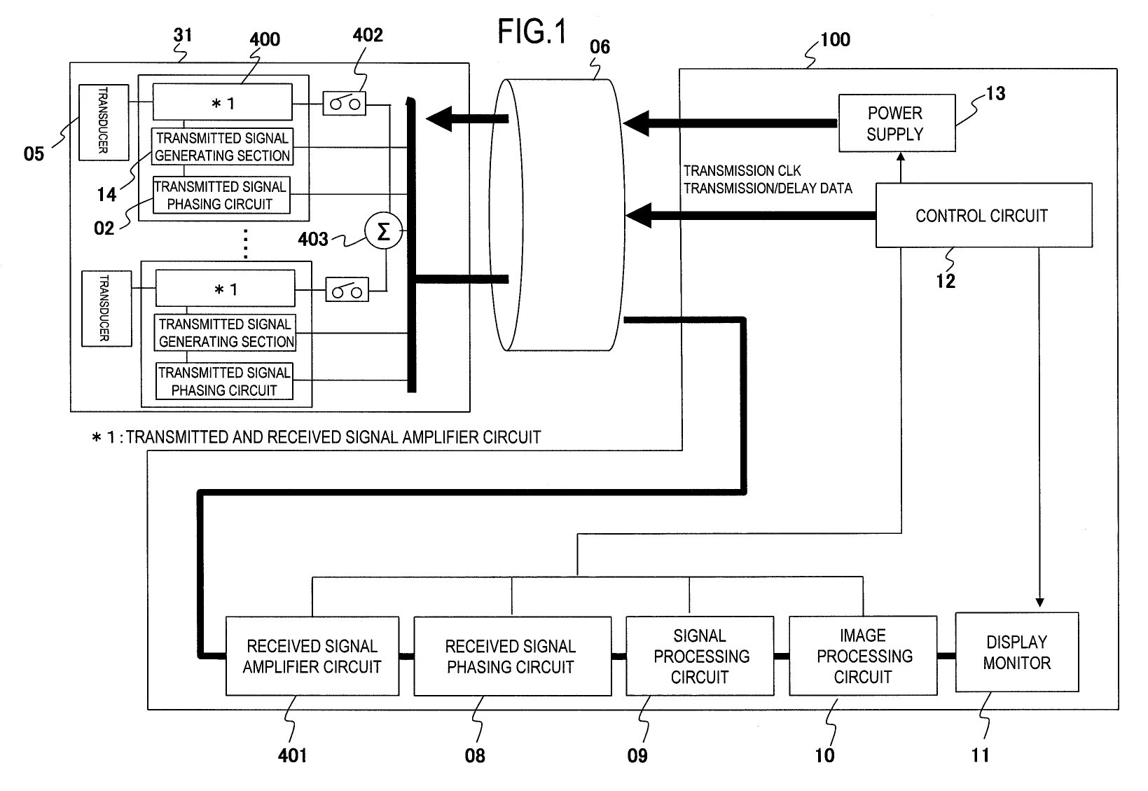

FIG. 1 shows an ultrasonic diagnostic apparatus according to a first embodiment.

The apparatus of the present embodiment is configured to include an ultrasonic probe 31, a transmitted signal phasing circuit 02, and a transmitted signal generating section 14, a transmitted and received signal amplifier circuit 400 with a transmission and reception separating function, a transducer 05, an ultrasonic probe cable 06, a received signal amplifier circuit 401, a received signal phasing circuit 08, a signal processing circuit 09, an image processing circuit 10, and a display monitor 11.

The ultrasonic probe 31 is characterized in that the transducer 05, the transmitted signal phasing circuit 02, the transmitted and received signal amplifier circuit 400, the transmitted signal generating section 14, a current adding switch 402, and a current adding circuit 403 are provided therein.

Electric signals after pulse signals or carrier signals of continuous waves are determined by the transmitted signal generating section 14 and amplified by the transmitted and received signal amplifier circuit 400 is applied to the transducer 05. The transducer 05 is formed to have a function of converting the signal into an ultrasonic wave and transmitting it to the subject and a function of receiving an ultrasonic wave reflected from the inside of the subject and converting the wave into an electric signal and outputting it.

The transmitted signal phasing circuit 02 is for adjusting an application timing of a transmitted signal to each driven transducer when forming a transmitted beam on the subject. Generally, a timing is controlled such that a voltage is applied early to a driven transducer in proportion to the distance of the transducer from the focusing position.

A transmitted signal amplifier section in the transmitted and received signal amplifier circuit 400 is for amplifying a transmitted signal waveform, which is formed by the transmitted signal generating section 14, up to the sufficient size by driving the transducer 05 in order to generate an ultrasonic signal.

The transmitted signal generating section 14 determines a carrier wave shape. For example, the transmitted signal generating section 14 includes a memory and stores one or plural carrier waveforms. The stored waveforms can be selected by a control circuit 12 in an ultrasonic diagnostic apparatus body 100. Alternatively, the waveform may be transmitted from the ultrasonic diagnostic apparatus body 100 through the ultrasonic probe cable 06 and stored whenever a transmitted signal is generated.

The current adding switch 402 is connected to a transducer in the relationship of 1:1, and the output destination is the current adding circuit 403. The current adding circuit 403 adds received signals of plural arbitrary elements as will be described in detail later, and the maximum number of output signals is the number of channels, in which phasing processing is possible, of the received signal phasing circuit 08 of the apparatus body 100.

Next, the received signal amplifier circuit 401 of the apparatus body 100 amplifies an ultrasonic signal acquired from the subject, and also has a function of changing the gain every time. When a gain sufficient to form a diagnostic image can be secured, the amplification function is not necessary in the transmitted and received signal amplifier circuit 400. In this case, the transmitted and received signal amplifier circuit 400 outputs a 1X signal or has only a function of changing the gain according to the depth.

The received signal phasing circuit 08 has a function of forming a beam similarly to the transmitted signal phasing circuit 02. The received signal phasing circuit 08 is for adjusting an addition timing of signals from all transducers, at which a signal from the subject is acquired, for each transducer when forming a received beam. A delay time is increased in proportion to the distance of a transducer from the focusing position for matching of addition timing of received signals of transducers far from the focusing position.

The signal processing circuit 09 and the image processing circuit 10 are for performing coordinate transformation processing according to the type of ultrasonic probe by performing signal processing for converting phased and added signals into brightness information by detection processing or the like and performing image signal processing represented by gamma (γ) processing or the like. Here, the processed signal is displayed as a diagnostic image on the display monitor 11.

In addition, each of the constituent circuits described above receives a basic clock signal from the control circuit 12, so that timing control of each section and the like are performed. Specifically, switching control of transmission and reception or switching of a diagnostic mode is performed. In addition, a power supply 13 is controlled by the control circuit 12 so as to output various electric power values. The electric power with various values generated herein is supplied to each circuit section (not shown).

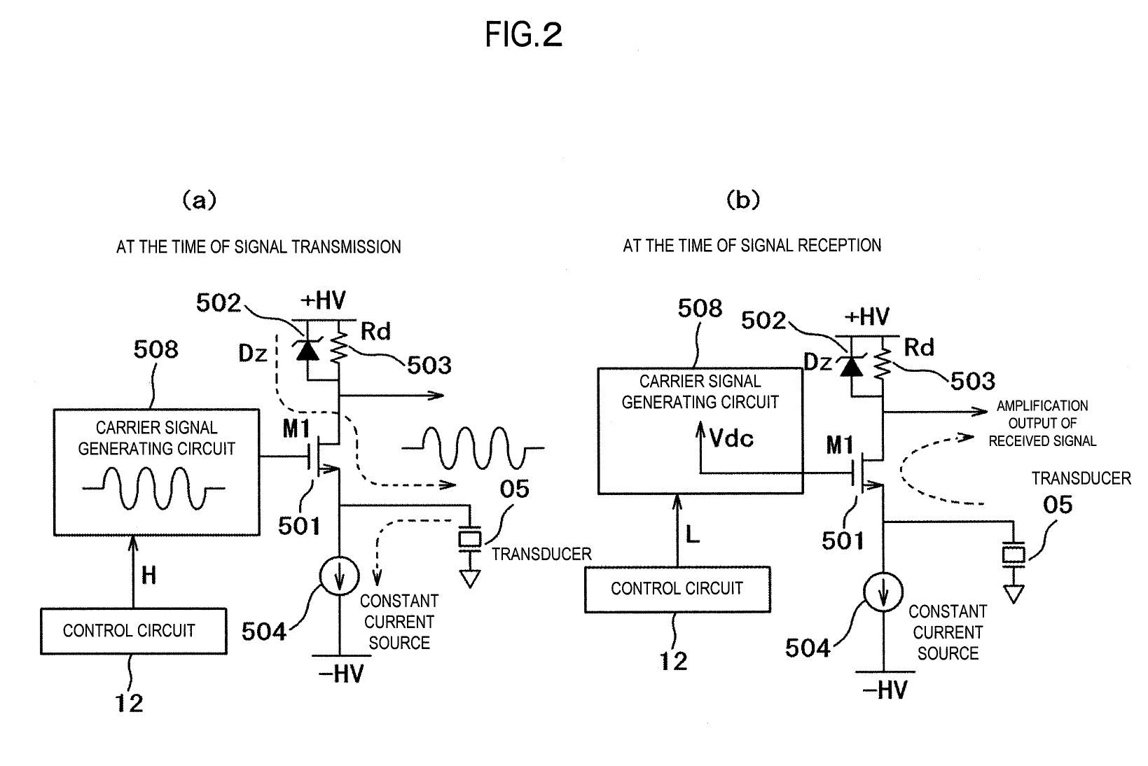

FIG. 2 shows a detailed circuit block diagram of an example of the transmitted and received signal amplifier circuit 400 which has both a transmission and reception separating function and a transmitted and received signal amplifying function associated with the present embodiment. In addition, (a) and (b) in FIG. 2 show states of the transmitted and received signal amplifier circuit 400 at the time of signal transmission and reception, respectively.

501, 502, 503, 504, 508, and 05 denote a driver element M1, a Zener diode Dz, a resistor Rd, a constant current source, a carrier signal generating circuit, and a transducer, respectively. The carrier signal generating circuit 508 is a block corresponding to the transmitted signal generating section 14 and the transmitted signal phasing circuit 02 shown in FIG. 1.

In the present embodiment, an N channel MOS field effect transistor (hereinafter, simply referred to as NMOSFET) is used as the driver element M1. The carrier signal generating circuit 508 is connected to a gate terminal of the driver element 501. The Zener diode 502 and the resistor 503 are connected in parallel between a drain terminal of the driver element 501 and a positive power supply +HV, and a received amplified signal is extracted from the drain terminal of the driver element 501 at the time of signal reception. The constant current circuit 504 is connected between a source terminal of the driver element 501 and a negative power supply −HV, and the source terminal of the driver element 501 is connected to the transducer 05 so that transmitted and received signals can be transmitted through the source terminal.

An operation at the time of signal transmission based on the circuit configuration shown in (a) of FIG. 2 is shown below. At the time of signal transmission, these circuits form a source follower. The voltage applied to the gate terminal of the driver element 501 appears at the source terminal as it is, driving the transducer 05. When a positive voltage is applied to the transducer 05, a current flows from the positive power supply +HV to the transducer 05 through the Zener diode 502 and the driver element 501. In addition, when a negative voltage is applied, a current flows from the transducer 05 to the constant current source 504.

The Zener diode 502 is provided to bypass a current when a large current of a transmitted signal flows, so that a voltage drop does not occur at the resistor 503. The Zener diode 502 may be replaced, for example, with a switch which becomes conductive at the time of signal transmission and non-conductive at the time of signal reception.

An operation at the time of signal reception in the circuit configuration shown in (b) of FIG. 2 is shown below. An N type high-voltage MOSFET (NMOSFET) forms a gate-grounded circuit at the time of signal reception. By controlling the carrier signal generating circuit 508 to output a constant value (Vdc), the gate potential of the driver element 501 is fixed and the driver element 501 operates as a gate-grounded amplifier. In this case, the gain is determined by a product of the transconductance gm of the field effect transistor and the resistance 503.

Thus, the received signal from the transducer 05 is amplified by the driver element 501 and is extracted as a received signal.

In addition, the carrier signal generating circuit 508 is controlled by the control circuit 12. For example, by the control circuit 12, the carrier signal generating circuit 508 is controlled to output a carrier signal when a signal with an output of H (high) is input and to output an appropriate DC electric potential (Vdc) when a signal with an output of L (low) is input. It is needless to say that such a circuit configuration can be easily formed by those skilled in the art.

The configuration of the present embodiment is advantageous in that the same driver element 501 can be used both for transmission and reception and a transmission and reception separating circuit, which is grounded for protection of the receiver circuit in the related art, can be omitted. As a result, it is possible to realize a reduction in the area and an improvement in the S/N ratio.

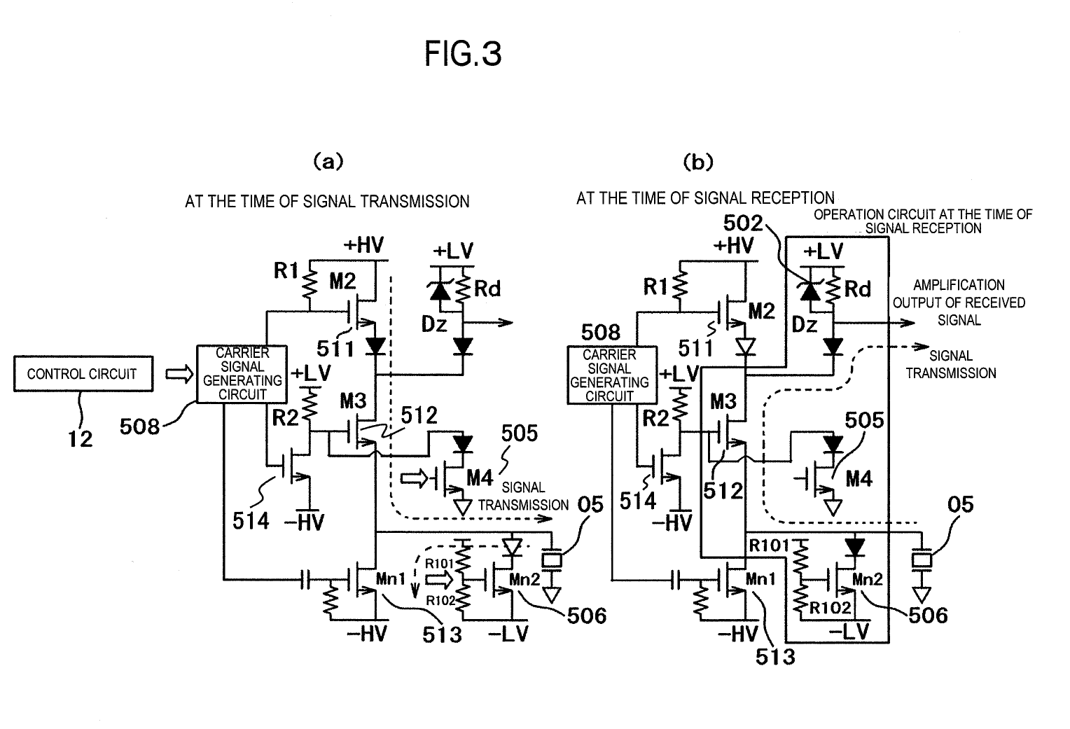

FIG. 3 shows a more specific example of the circuit configuration of the present embodiment. (a) and (b) of FIG. 3 show an operation of a circuit at the time of signal transmission and an operation of a circuit at the time of signal reception, respectively. In the present embodiment, since the power consumption is reduced by circuit switching at the time of signal transmission and reception, a configuration using a low-voltage transistor is possible. As a result, a smaller IC can be made. In addition, in FIGS. 3, 505, 506, 511, 512, 513, and 514 serve as driver elements formed by NMOSFETs, and 506 and 513 serve as current sources formed by NMOSFETs. In addition, the current source 513 is a current source using a differentiating circuit, and a current flows only when a signal is input.

Hereinafter, an operation at the time of signal transmission will be described. At the time of signal transmission, only the driver elements 511, 512, 513, and 514 are made to operate and the other driver elements 505 and 506 are turned OFF, as shown in (a) of FIG. 3. By forming the driver elements 511, 512, and 513 in multiple stages as shown in FIG. 3, a voltage applied to each transistor can be suppressed to be low. That is, since the driver elements 511, 512, and 513 can be formed by low-voltage transistors, a smaller IC can be made.

A transmission wave which is an. ON signal is applied to gates of the driver elements 511, 513, and 514 as a signal from the control circuit 12 and an OFF signal is applied to gates of the driver elements 505 and 506 as a signal from the control circuit 12 at the time of signal transmission shown in (a) of FIG. 3, that is, in a signal transmission section. For example, in the driver element 506, an electric potential lower than the source potential (−LV) of the driver element 506 is applied from the control circuit 12, so that the circuit does not operate.

At the time of signal reception shown in (b) of FIG. 3, that is, in a signal receiving section, an OFF signal is applied to the gates of the driver elements 511, 513, and 514 and, for example, a voltage equal to or higher than 3 V is applied to the gate of the driver element 505 to turn on the driver element. Accordingly, a fixed voltage is applied to the gate of the M3 by +LV and R2, and the M3 is gate-grounded. An electric potential determined by resistors R101 and R102 and −LV is applied to the driver element 506. Accordingly, the driver element 506 changes to an ON state and a current flows through the element.

As shown in (a) of FIG. 3, when a positive voltage is applied to the transducer 05, a current flows from a positive power supply to the transducer 05 through the driver elements 511 and 512. By outputting a carrier signal from the carrier signal generating circuit 508 to both the driver elements 511 and 514, the driver elements 511, 512, and 513 are made to operate simultaneously. In addition, when a negative voltage is applied to the transducer 05, a current is made to flow from the transducer 05 only at the falling edge by controlling the gate terminal of the driver element 513 through the differentiating circuit grounded to the output of the carrier signal generating circuit 508.

At the time of signal reception, only the driver elements 512, 505, and 506 are made to operate, and the driver elements 511, 513, and 514 are turned OFF by controlling the carrier signal generating circuit 508, as shown in (b) of FIG. 3. Thus, by circuit switching at the time of signal transmission and reception, the elements can be made to operate with low voltage supply ±LV at the time of signal reception. In this way, since the receiver circuit can be formed by lower voltage transistors, a smaller IC can be made.

In addition, since the gate potential of the driver element 512 is fixed by the driver element 505 at the time of signal reception, the input impedance is low. Accordingly, noise of the gate-grounded amplifier can be suppressed to be low.

In addition, in the circuit configuration shown in (a) of FIG. 3, an arrow ( [symbol] ) is given to a transistor to which a control signal from the control circuit 12 is actually output. However, the control method is not limited thereto, but any method may be used as long as it is a mechanism capable of turning a transistor ON and OFF.

### Second Embodiment

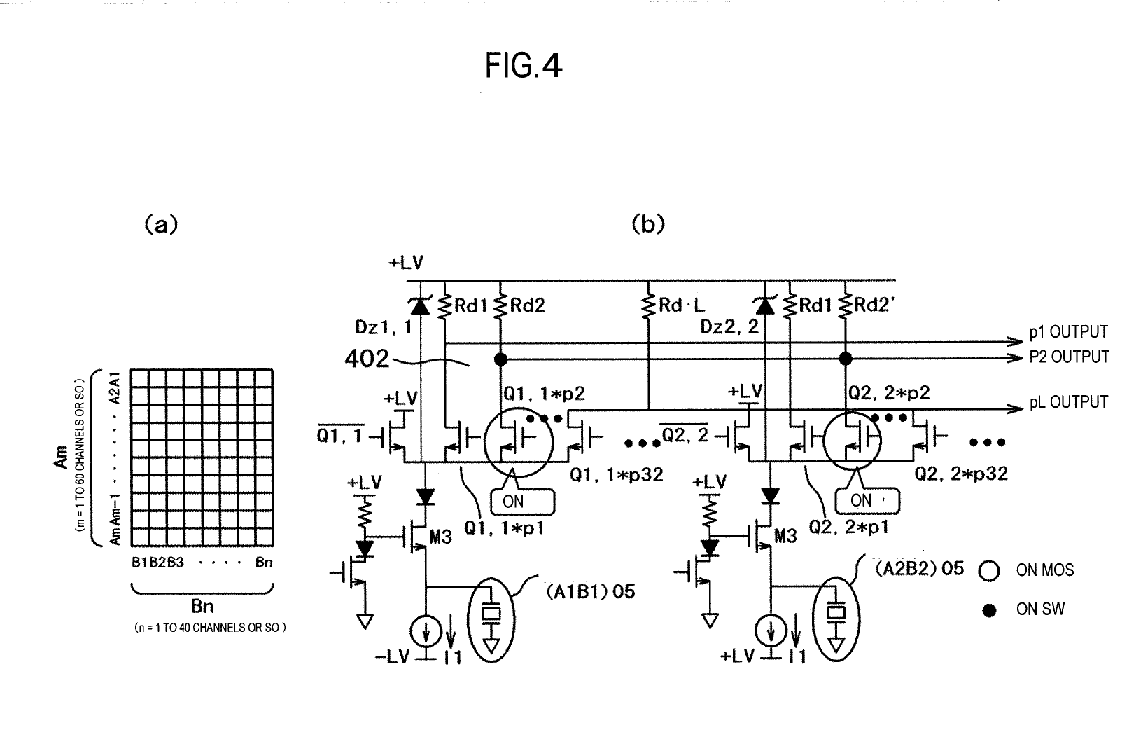

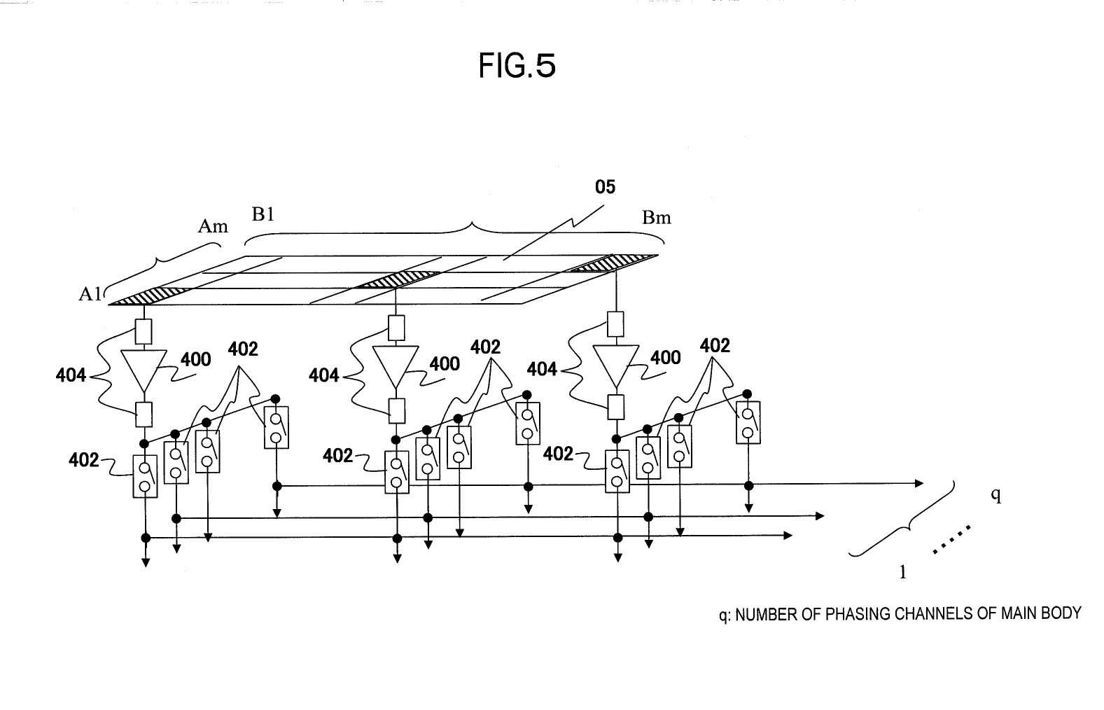

Next, as a second embodiment, an embodiment in which an analog matrix for adding a received signal from each cell for every arbitrary group at the time of wave receiving operation is added to the circuit configuration in the first embodiment will be described using FIGS. 4 and 5. (a) of FIG. 4 shows a transducer with a two-dimensional configuration of Am×Bn, and (b) of FIG. 4 shows a circuit configuration having an analog matrix switch (SW) 402 for reading a signal from an arbitrary element of the transducer Am×Bn.

In (a) of FIG. 4, a received signal is amplified by the gate-grounded transistor M3 and the resistor Rd. ADC constant current and a wave receiving AC current id flow through the M3, and an id*Rd signal is acquired in an Rd section.

In order to add received signals from the transducer (Am×Bn) 05 divided into plural arbitrary groups, a current matrix switch (SW) 402 is connected between the element M3 and the resistor Rd. Since a received signal can be extracted as a current from Rd, MOSFETs (Qm, n), the number of which is equivalent to the number of transducers, are connected to each output end of the current matrix switch group 402. However, one MOS may be provided at each connection point of the matrix, and may have a small size for switching only a received signal current.

(b) of FIG. 4 shows a state where received signals from two transducers A1B1 and A2B2 are added and input to P2 of a received signal phasing channel of the main body.

FIG. 5 shows a situation of group division in an operation at the time of signal reception of the transmitted and received signal amplifier circuit 400 when the transducer 05 forms an “Am×Bn” matrix array. The transmitted and received signal amplifier circuit 400 is connected to each transducer 05, and the current adding switch 402 is connected to the output. Switches, the number of which is equivalent to the number of received signal phasing channels of the main body (in the drawing, q channels), are connected to each output of the transmitted and received signal amplifier circuit 400, and switches corresponding to the number of transducers “Am×Bn” are connected to an output of each current adding switch.

A delay device 404 capable of arbitrarily delaying a received signal acquired by each transducer is provided before or behind the transmitted and received signal amplifier circuit 400. This delay device 404 can set an arbitrary amount of delay by the control circuit 12.

### Third Embodiment

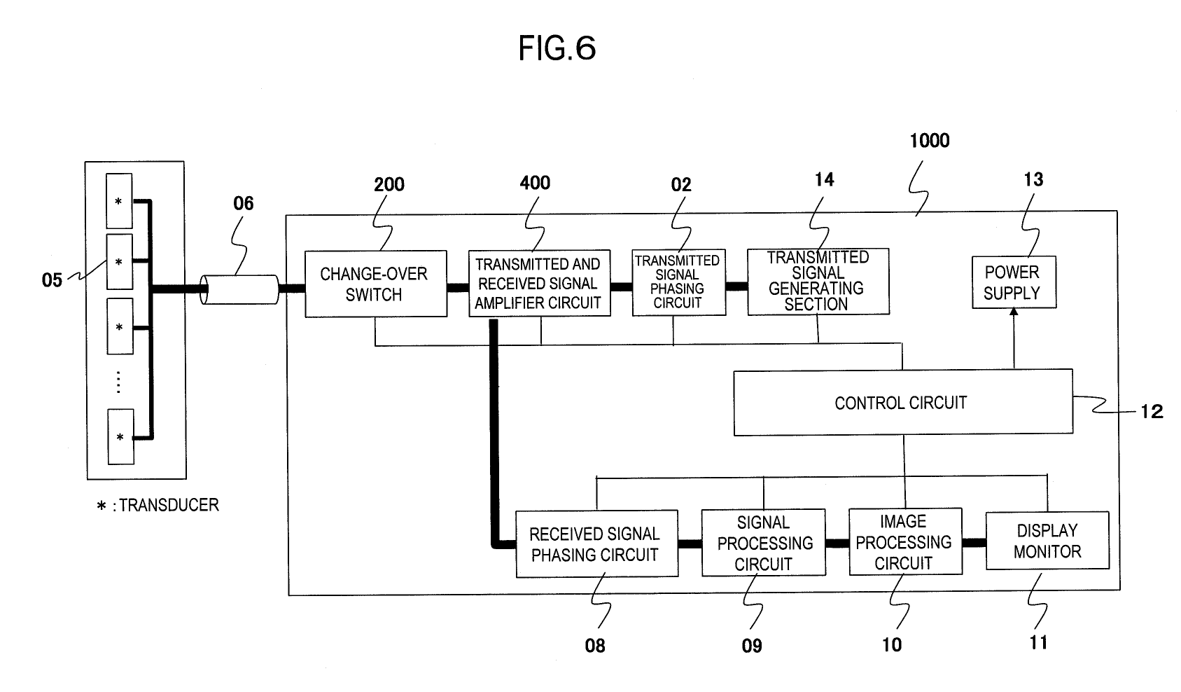

FIG. 6 shows, as a third embodiment, a case where the transmitted signal phasing circuit 02 and the transmitted and received signal amplifier circuit 400 described above are provided in the ultrasonic diagnostic apparatus body.

That is, the transmitted and received signal amplifier circuit 400 built in the ultrasonic probe 01, the transmitted signal generating section 14 corresponding to the carrier signal generating circuit 508, and the transmitted signal phasing circuit 02 in the first embodiment described above are mounted in an ultrasonic diagnostic apparatus body 1000. When the total number of transducers 05 in the ultrasonic probe 01 is different from the total number of transmitted and received signal amplifier circuits in the ultrasonic diagnostic apparatus, one channel of the transmitted and received signal amplifier circuit 400 may be electrically connected to plural different transducers by a change-over switch 200. In addition, the transmitted signal phasing circuit 02 may be omitted when necessary.

When the total number of transducers 05 and the total number of transmitted and received signal amplifier circuits 400 are equal, the change-over switch 200 is not necessarily needed.

In addition, the current adding switch 402 and the current adding circuit 403 are assumed to be included in the received signal phasing circuit 08 in FIG. 6. The current adding switch 402 and the current adding circuit 403 perform received signal phasing processing, and one object of the current adding switch 402 and the current adding circuit 403 is to prevent an increase in the number of received signals of the ultrasonic probe cable 06 by adding received signals of plural arbitrary elements when the total number of transducers 05 is larger than the total number of transmitted and received signal amplifier circuits 400 in the previous embodiment. However, in the method in which the transmitted and received signal amplifier circuit 400 is included in FIG. 6, the number of receiving signal lines used and the total number of transmitted and received signal amplifier circuits 400 are generally equal in the ultrasonic probe cable 06. Accordingly, previous received signal addition is performed by the received signal phasing circuit 08. The current adding switch 402 and the current adding circuit 403 may also be used for this phasing method.

### INDUSTRIAL APPLICABILITY

According to the invention, the configuration of an amplifier circuit used for both transmission and reception which has a function of adding currents of received signals from plural elements becomes possible in the ultrasonic diagnostic apparatus. Therefore, since the same transistor element can be used both for transmission and reception, it is very useful in that it is possible to realize a reduction in the area and an improvement in the S/N ratio.

### Reference Signs List

01, 31: ultrasonic probe
02: transmitted signal phasing circuit
03: transmitted signal amplifier circuit
04: transmission and reception separating circuit
05: transducer
06: ultrasonic probe cable
07: received signal amplifier circuit
08: received signal phasing circuit
09: signal processing circuit
10: image processing circuit
11: display monitor
12: control circuit
13: power supply
100, 1000: ultrasonic diagnostic apparatus body
200: change-over switch
400: transmitted and received signal amplifier circuit
401: received signal amplifier circuit
402: current adding switch
403: current adding matrix
404: delay device

1. An ultrasonic diagnostic apparatus comprising:
an ultrasonic probe having a plurality of transducers;
an apparatus body for reception processing of received signals from the plurality of transducers;
a transmitted and received signal amplifier circuit which amplifies signals transmitted to the plurality of transducers and amplifies received signals from the plurality of transducers; and
a current adding circuit which adds currents of the received signals amplified by the transmitted and received signal amplifier circuit.

2. The ultrasonic diagnostic apparatus according to claim 1, further comprising:
a current adding switch which connects the transmitted and received signal amplifier circuit and the current adding circuit to each other.

3. The ultrasonic diagnostic apparatus according to claim 2,
wherein the transmitted and received signal amplifier circuit, the current adding circuit, and the current adding switch are built in the ultrasonic probe.

4. The ultrasonic diagnostic apparatus according to claim 1,
wherein the transmitted and received signal amplifier circuit includes an FET element operating as a source follower circuit at the time of signal transmission.

5. The ultrasonic diagnostic apparatus according to claim 1,
wherein the transmitted and received signal amplifier circuit includes an FET element operating as a gate-grounded circuit at the time of signal reception.

6. The ultrasonic diagnostic apparatus according to claim 2,
wherein the current adding switch is formed by one FET element corresponding to each of the transducers.

7. The ultrasonic diagnostic apparatus according to claim 4,
wherein the circuit using the FET element operating as the source follower circuit connects a plurality of FETs to each other.

8. The ultrasonic diagnostic apparatus according to claim 2,
wherein the transmitted and received signal amplifier circuit, the current adding circuit, and the current adding switch are provided in the apparatus body.

9. The ultrasonic diagnostic apparatus According to claim 4,
wherein the transmitted and received signal amplifier circuit includes an FET element operating as a gate-grounded circuit at the time of signal reception.

10. An ultrasonic probe comprising:
a plurality of transducers;
a transmitted and received signal amplifier circuit which amplifies signals transmitted to the plurality of transducers and amplifies received signals from the plurality of transducers; and
a current adding circuit which adds currents of the received signals amplified by the transmitted and received signal amplifier circuit.

11. The ultrasonic probe according to claim 10, further comprising:
a current adding switch which connects the transmitted and received signal amplifier circuit and the current adding circuit to each other.

12. The ultrasonic probe according to claim 10,
wherein the transmitted and received signal amplifier circuit includes an FET element operating as a source follower circuit at the time of signal transmission.

13. The ultrasonic probe according to claim 10,
wherein the transmitted and received signal amplifier circuit includes an FET element operating as a gate-grounded circuit at the time of signal reception.

14. The ultrasonic probe according to claim 13,
wherein the current adding switch is formed by one FET element corresponding to each of the transducers.

15. The ultrasonic probe according to claim 12,
wherein the transmitted and received signal amplifier circuit includes an FET element operating as a gate-grounded circuit at the time of signal reception.

16. An ultrasonic diagnostic method comprising:
amplifying signals transmitted to a plurality of transducers in an ultrasonic probe;
transmitting the amplified transmitted signals to the transducers;
transmitting ultrasonic waves from the transducers on the basis of the transmitted signals;
receiving ultrasonic waves from the transducers;
generating the received signals of the transducers on the basis of the received ultrasonic waves; and
adding currents on the basis of the received signals.
